(19) United States
(12) Patent Application Publication
(10) Pub. No.: US 2017/0251988 A1
(43) Pub. Date: Sep. 7, 2017

(54) ULTRASOUND IMAGING APPARATUS

(71) Applicant: KONINKLIJKE PHILIPS N.V., EINDHOVEN (NL)

(72) Inventors: FRANK MICHAEL WEBER, EINDHOVEN (NL); THOMAS HEIKO STEHLE, Eindhoven (NL); IRINA WAECHTER-STEHLE, EINDHOVEN (NL); JUERGEN WEESE, EINDHOVEN (NL)

(21) Appl. No.: 15/510,103

(22) PCT Filed: Sep. 11, 2015
(86) PCT No.: PCT/EP2015/070806

§ 371 Date: Mar. 9, 2017

(30) Foreign Application Priority Data
Sep. 18, 2014 (EP) 14185262.4

(51) Int. Cl. A61B 6/12; G06T 7/00; A61B 8/00; A61B 6/00; A61B 8/12; A61B 8/08

(52) U.S. Cl. CPC A61B 6/12; A61B 8/12; A61B 8/08; A61B 8/4245; A61B 8/483; A61B 8/0883; A61B 6/466; A61B 8/523; G06T 7/0012

(57) ABSTRACT

An ultrasound imaging apparatus (10) for providing ultrasound images of a patient (12) is disclosed. The imaging apparatus (10) comprises an ultrasound acquisition unit (14) for acquiring ultrasound data (42) of a patient's body in a field of view (16), a position determining unit (24) for determining a position (26) within the patient's body. An ultrasound data transformation unit (30) is provided for transforming the ultrasound data in the filed of view on the basis of the determined position to transformed ultrasound data (42) in a virtual field of view (20) having a virtual viewing direction (28) different from the viewing direction of the ultrasound acquisition unit.

### FIELD OF THE INVENTION

The present invention relates to an ultrasound imaging apparatus for providing ultrasound images of a patient. The present invention further relates to an ultrasound imaging method for providing ultrasound images of a patient and a computer program comprising program code means for causing a computer to carry out steps of the method when said computer program is carried out on a computer.

### BACKGROUND OF THE INVENTION

In the field of medical imaging systems it is generally known to use catheters including ultrasound echo probes for providing an ultrasound view from a position within the patient's body, such as e.g. intracardiac echocardiography. A corresponding ultrasound catheter echo probe for providing intracardiac ultrasound images is e.g. known from U.S. Pat. No. 8,270,694 B2.

The ultrasound catheters including ultrasound echo probes are expensive and have to be introduced into the patient's body so that the examination is complicated, time consuming and probably risky for the patient. Since not all examinations of a patient require a catheter including an ultrasound echo probe, the use of these catheters can be omitted, however, an internal view within the patient's body from a catheter probe position may be helpful for the operator to analyze the ultrasound images and the compare the results with other ultrasound images.

US 2013 0223702 A1 discloses a surgical instrument navigation system that visually simulates a virtual volumetric scene of a body cavity of a patient from a point of view of a surgical instrument residing in the cavity of the patient, wherein the surgical instrument may be a steerable surgical catheter with a biopsy device and/or a surgical catheter with a side-exiting medical instrument.

### SUMMARY OF THE INVENTION

It is therefore an object of the invention to provide an ultrasound imaging apparatus and an ultrasound imaging method which provides an internal view corresponding to a view from an ultrasound catheter probe position with low technical effort. According to one aspect an ultrasound imaging apparatus for providing ultrasound images of a patient is provided, comprising:

an ultrasound acquisition unit for acquiring ultrasound data of a patient's body in a field of view,

a position determining unit for determining a position within the patient's body, and

an ultrasound data transformation unit for transforming the ultrasound data in the filed of view on the basis of the determined position to transformed ultrasound data in a virtual field of view having a virtual viewing direction different from the viewing direction of the ultrasound acquisition unit,

wherein the position determining unit is adapted to determine the position and/or the virtual viewing direction of the virtual field of view on the basis of the ultrasound data or on the basis of X-ray images provided by an X-ray unit.

According to another aspect an ultrasound imaging method for providing ultrasound images of a patient is provided, comprising the steps of:

receiving ultrasound data of a patient's body in a field of view,

determining a position within the patient's body,

transforming the ultrasound data in the field of view on the basis of the determined position to transformed ultrasound data in a virtual field of view having a virtual viewing direction different from the viewing direction of the field of view, and

determining the position and/or the virtual viewing direction of the virtual field of view on the basis of the ultrasound data or on the basis of X-ray images provided by an X-ray unit.

According to another aspect a computer program is provided comprising program code means for causing a computer to carry out the steps of the method according to the invention when said computer program is carried out on the computer.

Preferred embodiments are defined in the dependent claims. It shall be understood that the claimed method has similar and/or identical preferred embodiments as the claimed device and as defined in the dependent claims.

The present invention is based on the idea to acquire ultrasound data of a patient by means of an ultrasound acquisition unit and to transform the ultrasound data in the field of view as captured to ultrasound data in a virtual field of view corresponding to a position within the patient's body determined by the position determining unit. The virtual field of a view has a virtual viewing direction as seen from the position within the patient's body determined by the position determining unit so that an internal view can be derived from the ultrasound data acquired by the ultrasound acquisition unit. Hence, the internal view within the patient's body can be provided without introducing a catheter into the patient's body including an ultrasound echo probe merely by transforming the ultrasound data from the real field of view of the ultrasound acquisition unit to the virtual field of view. Hence, the technical effort for providing ultrasound images from an internal view of the patient's body can be reduced.

In a preferred embodiment, the position is a position of a catheter probe within the patient's body determined by the position determining unit. In other words, the position determining unit is adapted to determine a position of a catheter probe within the patient's body as the position on the basis of which the virtual field of view is determined. This is a possibility to precisely determine a position of interest in the patient's body by means of a catheter, wherein the use of an expensive catheter ultrasound echo probe can be omitted.

In a preferred embodiment, the position determining unit is further adapted to determine an orientation of the catheter probe within the patient's body, wherein the virtual viewing direction is determined on the basis of the orientation of the catheter probe. This is a possibility to provide an ultrasound image in the virtual viewing direction corresponding to a viewing direction of the catheter probe without the need of a catheter having an ultrasound echo probe. Hence, the ultrasound images of an echo probe can be virtually simulated.

The position determining unit is adapted to determine the position and/or the virtual viewing direction of the virtual field of view on the basis of the ultrasound data. This is a possibility to identify anatomical features of the patient or a catheter probe within the patient's body in order to precisely determine the relevant position from which images in the virtual viewing direction are required.

The position determining unit is connected to the X-ray unit providing X-ray images of the patient's body, wherein the position determining unit is adapted to determine the position and/or the virtual viewing direction of the virtual field of view on the basis of the X-ray images. This is a possibility to further improve the determination of the position within the patient's body, since X-ray as a different analysis method is utilized. In a further preferred embodiment, the X-ray unit is used to determine the position and the orientation of the catheter probe. This is a possibility to determine the catheter probe with high precision.

In a further preferred embodiment, the virtual field of view is determined as a virtual viewing direction from the determined position. This is a possibility to simulate the acquisition of ultrasound images by a catheter ultrasound echo probe.

In a preferred embodiment, the position determining unit comprises a segmentation unit for segmenting the ultrasound data and for providing segmentation data, wherein the position and/or the virtual viewing direction of the field of view is determined on the basis of the segmentation data. This is a possibility to further improve the determination of the position within the patient's body, since the ultrasound data can be analyzed e.g. for anatomical features so that the position within the patient's body can be precisely determined within the anatomical context.

In a further preferred embodiment, the position determining unit is adapted to determine the virtual viewing direction on the basis of anatomical features identified in the segmentation data. This is a possibility to define the virtual viewing direction with respect to identified anatomical features and organs so that a predefined or a standard view of certain anatomical features can be automatically determined.

In a preferred embodiment, the position determining unit comprises an input device for determining the position and the direction of the virtual viewing direction on the basis of a user input. This is a possibility to flexibly determine the position and the direction of the virtual viewing directing by the user so that an arbitrary viewing direction can be selected.

In a preferred embodiment, the input device is adapted to determine the position in the ultrasound data received from the ultrasound acquisition unit. This is a possibility to improve the comfort for the user, since the position can be determined in the ultrasound images, e.g. by means of a mouse click or the like so that the position can be determined precisely with low technical effort.

In a preferred embodiment, the imaging apparatus comprises a display unit for displaying the transformed ultrasound data in the virtual viewing direction. This is a possibility to provide ultrasound images corresponding to the determined internal virtual viewing direction.

In a further preferred embodiment, the ultrasound acquisition unit is an external ultrasound acquisition unit located outside the patient's body or a catheter based ultrasound acquisition unit. This is a possibility to reduce the technical effort, since different ultrasound acquisition units can be utilized for acquiring the ultrasound data and the ultrasound data can be transformed in order to provide a corresponding ultrasound image in the virtual viewing direction from the position within the patient's body.

In a preferred embodiment, the ultrasound data comprises a plurality of voxels each including an ultrasound measurement value, wherein the transformation unit is adapted to transform the ultrasound measurement values of the voxels in the field of view to voxels of the virtual field of view. This is a possibility to transform the ultrasound data of the field of view with low technical effort to the transformed ultrasound data in the virtual field of view, since each voxel can be transformed to a voxel of the virtual field of view with e.g. by means of a transformation matrix.

As mentioned above, the position within the patient's body can be determined in order to define the virtual field of view in order to simulate the acquisition of ultrasound data by means of a catheter ultrasound echo probe. The position on the basis of which the virtual field of view is determined can be defined by determining a position of a real catheter probe within the patient's body, e.g. by means of a tracking unit or within the ultrasound image or an X-ray image, the position can be determined on the basis of the anatomical context in the patient's body by segmenting the ultrasound data and by determining organs within the patient's body on the basis of the segmentation data or by means of a combination of the catheter tracking and the anatomical context. In a further embodiment, the position within the patient's body can be determined by means of a manual user input so that the position can be defined flexibly as desired. This is in general a possibility to improve the ultrasound imaging analysis, since each view within the patient's body can be determined with low technical effort merely by transforming the acquired ultrasound data to a virtual field of view.

### BRIEF DESCRIPTION OF THE DRAWINGS

These and other aspects of the invention will be apparent from and elucidated with reference to the embodiment(s) described hereinafter. In the following drawings

FIG. 1 shows a schematic representation of an ultrasound imaging apparatus in use to scan a volume of a patient's body and to transform the field of view to a virtual field of view;

FIG. 2 shows a schematic image of a catheter probe within the patient's body defining a position of a virtual field of view;

FIG. 3 shows an ultrasound image and segmented organs within the ultrasound image for determining the position of the virtual field of view;

FIG. 4 shows an ultrasound image in the field of view and a transformed ultrasound image in the virtual field of view; and

FIG. 5 shows a schematic flow diagram of a method for providing ultrasound images in a virtual field of view from a position within the patient's body.

### DETAILED DESCRIPTION OF THE INVENTION

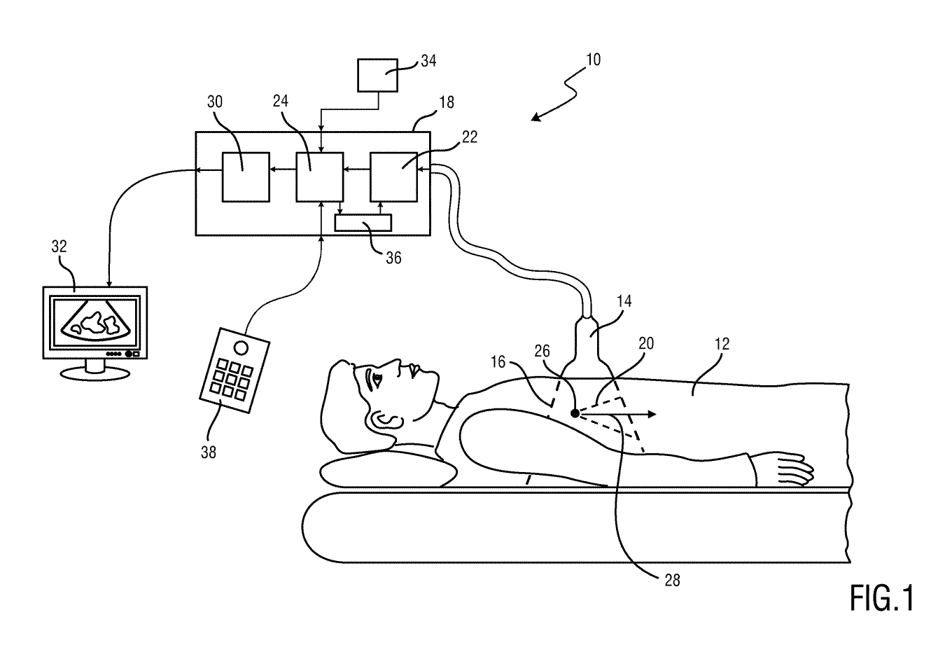

FIG. 1 shows a schematic illustration of an ultrasound imaging apparatus 10 according to one embodiment. The ultrasound imaging apparatus 10 is applied to inspect a volume of an anatomical side, in particular an anatomical side of a patient 12. The ultrasound imaging apparatus comprises an ultrasound acquisition unit 14 in particular an ultrasound probe 14 having at least one transducer array including a multitude of transducer elements for transmitting and receiving ultrasound waves. The transducer elements are preferably arranged in a 2D array for providing 3D ultrasound image data. The ultrasound acquisition unit 14 acquires ultrasound data in a field of view 16 within the patient's body and provides corresponding 3D ultrasound data.

The ultrasound imaging apparatus 10 comprises in general an image processing apparatus 18 for evaluating the ultrasound data received from the ultrasound acquisition unit 14 and for transforming the ultrasound data in the field of view 16 to a virtual field of view 20 as described in the following.

The ultrasound acquisition unit 14 may be an external ultrasound acquisition unit which is located entirely outside the patient's body or may be a catheter probe inserted into the patient's body, wherein the acquisition unit provides e.g. a transesophageal echocardiogram (TEE) or a transthoracic echocardiogram (TTE) as the ultrasound image.

The image processing apparatus 18 comprises an image evaluation unit 22 connected to the ultrasound acquisition unit 14 for evaluating the ultrasound data and for providing ultrasound image data from the volume or object of the patient which is analyzed by the ultrasound acquisition unit 14 in the field of view 16. The image processing apparatus 18 further comprises a position determining unit 24, which is adapted to determine a position 26 within the patient's body. The position determining unit 24 is further adapted to determine the virtual field of view 20 as a virtual cone from the determined position 26 in a virtual viewing direction 28. The position determining unit 24 is connected to the image evaluation unit 22 and receives the ultrasound data from the image evaluation unit 22 of the field of view 16 and determines the position 26 preferably within the field of view 26 of the ultrasound acquisition unit 14. The position determining unit 24 further determines the virtual field of view 20 on the basis of the position 26 and the virtual viewing direction 28 e.g. having a predefined or selectable viewing angle so that a volume within the virtual field of view 20 can be determined.

The image processing apparatus 18 further comprises a transformation unit 30 for transforming the ultrasound data in the field of view 16 to transformed ultrasound data in the virtual field of view. The transformed ultrasound data is provided to a display unit 32 for displaying the transformed ultrasound data in the virtual field of view 20.

The transformation unit 30 receives the ultrasound data as a 3D array of voxels each including an ultrasound measurement value and transforms the voxels of the field of view 16 to voxels of the virtual field of view 20 in the virtual viewing direction 28 so that the transformed ultrasound data can be provided and displayed on a display unit 32 as if the transformed ultrasound data would have been acquired by an ultrasound probe located at the position 26 and directed in the virtual viewing direction 28.

The position 26 within the patient's body and the virtual viewing direction 28 can be determined in different ways. The position 26 and the virtual viewing direction 28 may be determined as a position of a catheter introduced in the patient's body so that the virtual field of view 20 can be determined as if the transformed ultrasound data would have been acquired by means of the catheter ultrasound probe as described in the following. The position of the catheter may be determined by an electromagnetic tracking unit, by means of the ultrasound acquisition unit 14 or by means of an X-ray unit 34 which may be connected to the ultrasound imaging apparatus 10 and to the position determining unit 24 e.g. by means of pattern detection.

The ultrasound imaging apparatus 10 may further comprise a segmentation unit 36 connected to the image evaluation unit 22 and to the position determining unit 24, wherein the segmentation unit 24 provides segmentation data on the basis of the ultrasound data and determines anatomical features within the field of view 16. The position determining unit 24 can identify on the basis of the segmentation data different anatomical features and/or organs within the field of view 16 and determines the virtual field of view 20 on the basis of the segmentation data. This is a possibility to automatically define the virtual field of view 20 in the direction of a certain anatomical feature to be examined or which corresponds to a usual field of view of a catheter ultrasound probe during corresponding catheter examinations.

The ultrasound imaging apparatus 10 may further be connected to or may further comprise an input device 38 which is provided for a user input to determine the position 26 and/or the virtual viewing direction 28 in the patient's body so that the virtual field of view 20 can be individually determined by the user. The user may identify the position and the virtual viewing direction 28 within the ultrasound data or within the X-ray data or may determine the position on the basis of the segmentation data so that the virtual field of view 20 can be individually determined with high precision by the user.

In general, the ultrasound imaging apparatus 10 can provide the transformed ultrasound data in the virtual field of view 20 as if a catheter including an ultrasound echo probe would have been used and is located at the position 26, wherein the use of such a catheter probe can be omitted.

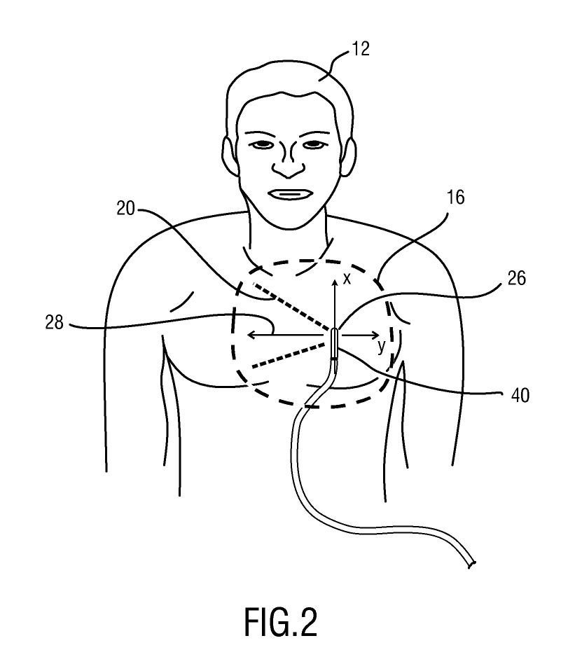

FIG. 2 shows an embodiment of the ultrasound imaging apparatus 10. In this embodiment, a catheter probe 40 is introduced into the patient's body 12 and the position determining unit 24 determines a spatial position of the catheter probe 14 as the position 26 in order to determine the virtual field of view 20.

The position determining unit 24 determines the position of the catheter probe 14 by means of an electromagnetic tracking unit, by means of the X-ray device 34 or by means of the ultrasound acquisition unit 14 which provides the ultrasound data from the field of view 16, in which the catheter probe 40 is located. The position determining unit 24 is also adapted to determine an orientation of the catheter probe 40 within the patient's body 12 in order to determine the position 26 and the virtual viewing direction 28 on the basis of the position and orientation of the catheter probe 40. The transformation unit 30 transforms the ultrasound data of the ultrasound acquisition unit 14 in the field of view 16 to the virtual field of view 20 and displays the transformed ultrasound data at the display unit 32 so that an ultrasound image can be displayed as if the transformed ultrasound data would have been captured by means of the catheter probe 40.

A preferred application of the ultrasound imaging apparatus is the ultrasound examination of the heart of the patient 12. The ultrasound acquisition unit 14 may be an ultrasound probe disposed outside the patient's body e.g. attached to the skin of the thorax for acquiring the ultrasound data or may be an ultrasound catheter introduced into the patient's body 12 e.g. into the esophagus for acquiring the ultrasound data of the patient 12.

Hence, ultrasound images from an internal view within the patient's body can be provided without the use of a catheter having an ultrasound echo probe.

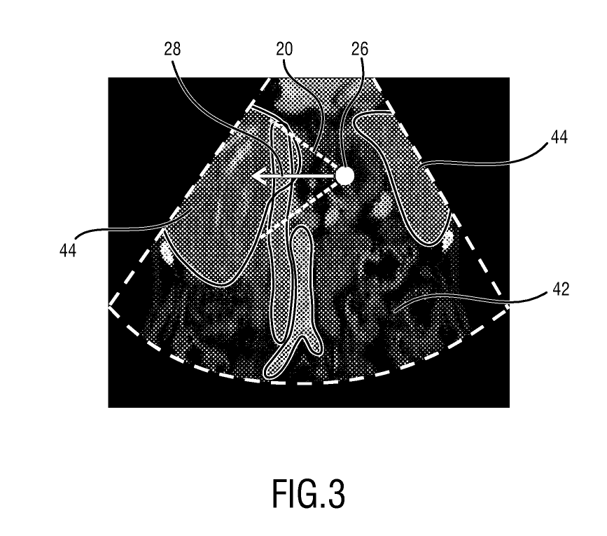

FIG. 3 shows an embodiment of the determination of the position 26 and the virtual viewing direction 28. In this embodiment, the segmentation unit 36 segments different organs in the ultrasound data 42 captured by the ultrasound acquisition unit 24 and provides segmentation data 44 of the different organs or anatomical features of the patient 12. The position determining unit 24 determines the position 26 and the virtual viewing direction 28 on the basis of the segmentation data 44 and the correspondingly identified organs and/or anatomical features so that the organs or anatomical features of interest are within the virtual field of view 20 or the virtual cone and correspondingly displayed in the transformed ultrasound data on the display unit 32. Hence, the organs and/or anatomical features of interest can be automatically displayed as if a catheter including an ultrasound echo probe would be located at the position 26 and directed correspondingly in the virtual viewing direction 28 to scan the respective organs and/or anatomical features.

It shall be understood that the embodiments of FIGS. 2 and 3 can be combined in one embodiment so that the position and the virtual viewing direction 28 is determined based on the identified position of the catheter probe 40 and on the basis of the segmentation data 44 provided by the segmentation unit 36. In a certain embodiment, the position 26 can be determined on the basis of the detected position of the catheter probe 40 and the virtual viewing direction 28 can be determined on the basis of the segmentation data 44 so that the relevant organs and/or anatomical features can be displayed automatically from the position of the catheter probe 40.

It shall be understood that the user input may be utilized for adjusting the position 26 and the viewing direction 28 determined on the basis of the position of the catheter probe 40 and on the basis of the segmentation data 44.

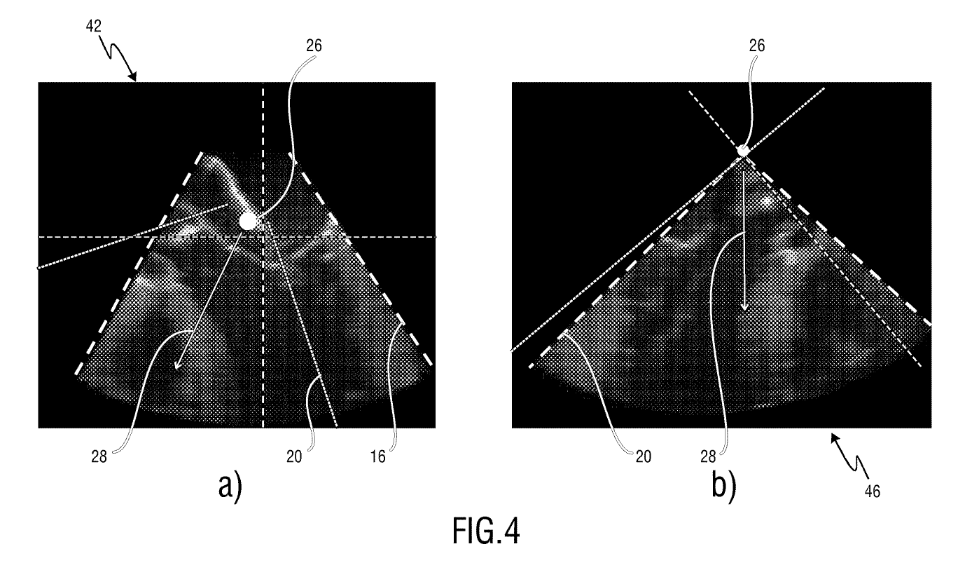

FIG. 4 shows ultrasound data in the field of view 16 and transformed ultrasound data in the virtual field of view 20 transformed by the transformation unit 30. FIG. 4a shows the ultrasound data 42 captured by the ultrasound acquisition unit 14 in the field of view 16 including the position 26, the virtual viewing direction 28 and the virtual field of view 20. On the basis of the position 26 and the virtual viewing direction 28, the ultrasound data 42 is transformed to transformed ultrasound data 46 shown in FIG. 4b. The transformed ultrasound data 46 is displayed in the virtual field of view 20 seen from the position 26 in the virtual viewing direction 28 as if the transformed ultrasound data 46 would have been captured from the position 26 within the patient's body 12. Hence, the use of a catheter including an ultrasound echo probe can be simulated by transforming the ultrasound data 42 in the field of view 16 to the transformed ultrasound data 46 in the virtual field of view 20.

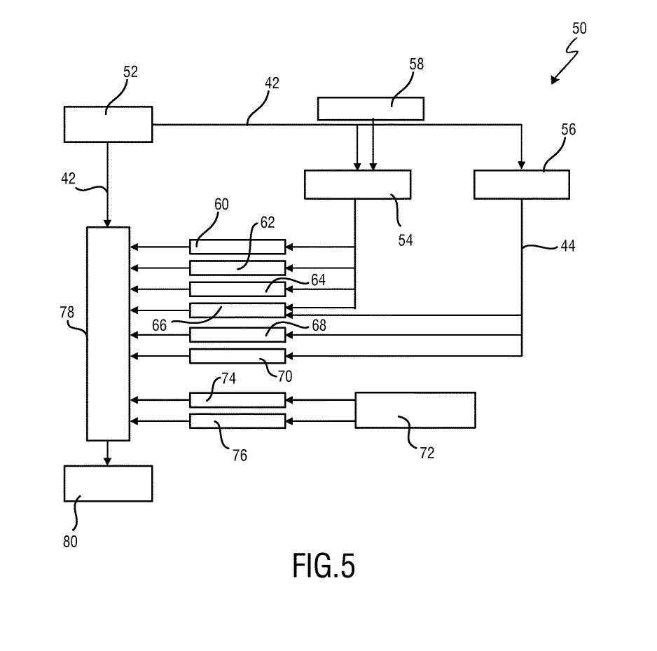

FIG. 5 shows a schematic block diagram of an ultrasound imaging method for providing ultrasound images of the patient 12 generally denoted by 50. The method 50 starts with acquiring 3D ultrasound data from the patient 12 by means of the ultrasound acquisition unit 14 as shown at a step 52. The ultrasound data 42 may be formed as a transthoracic echocardiogram (TTE) or as a transesophageal echocardiogram (TEE) of the patient 12. The ultrasound data 42 can be provided to the position determining unit 24 as shown at step 54 additionally or alternatively, the ultrasound data 42 can be provided to the segmentation unit 36 as shown at 56. The X-ray unit 34 acquires X-ray data as shown at 58 and provides the X-ray data to the position determining unit 24 as shown at 54.

In one embodiment, the position determining unit 24 determines the position 26 as shown at 60 and the virtual viewing direction 28 as shown at 62 on the basis of the ultrasound data 42 or the X-ray data.

Alternatively, the position determining unit 24 determines the virtual position 26 on the basis of the ultrasound data and/or the X-ray data as shown at 64 and the virtual viewing direction 28 on the basis of the ultrasound data 42 and/or the X-ray data and additionally on the basis of the segmentation data 44 provided by the segmentation unit 36 as shown at 66.

In an alternative embodiment, the position determining unit 24 is adapted to determine the position 26 and the virtual viewing direction 28 merely on the basis of the segmentation data 44 provided by the segmentation unit 36 as shown at 68 and 70.

Alternatively, a user input is provided by means of the input device 38 as shown at 72 and the position determining unit 24 is adapted to determine the position 26 on the basis of the user input as shown at 74 and the virtual viewing direction on the basis of the user input as shown at 76.

The transformation unit 30 transforms the ultrasound data 42 in the field of view 16 to the transformed ultrasound data 46 in the virtual field of view 20 as shown at 78 and provides the transformed ultrasound data 46 to the display unit 32 for displaying the transformed ultrasound data 46 in the virtual field of view 20 as if the ultrasound data 46 would have been acquired from the position 26 within the patient's body 12. The transformed ultrasound data 46 is provided to the display unit 32 for displaying the transformed ultrasound data as shown at 80.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. An ultrasound imaging apparatus for providing ultrasound images of a patient, comprising:
an ultrasound acquisition unit for acquiring ultrasound data of a patient's body in a field of view,
a position determining unit for determining a position within the patient's body, and
an ultrasound data transformation unit for transforming the ultrasound data in the filed of view on the basis of the determined position to transformed ultrasound data in a virtual field of view having a virtual viewing direction different from the viewing direction of the ultrasound acquisition unit,
wherein the position determining unit comprises a segmentation unit for segmenting the ultrasound data and for providing segmentation data,
wherein the position determining unit is adapted to determine the position on the basis of the ultrasound data, or on the basis of the segmentation data, or on the basis of X-ray data provided by an X-ray unit, and to determine the virtual viewing direction merely on the basis of anatomical features identified on the basis of the segmentation data or additionally on the basis of the ultrasound data or the X-ray data.

2. The ultrasound imaging apparatus as claimed in claim 1, wherein the position is a position of a catheter probe within the patient's body determined by the position determining unit.

3. The ultrasound imaging apparatus as claimed in claim 2, wherein the position determining unit is further adapted to determine an orientation of the catheter probe within the patient's body, wherein the virtual viewing direction is determined on the basis of the orientation of the catheter probe.

4. The ultrasound imaging apparatus as claimed in claim 1, wherein the virtual field of view is determined as a virtual viewing direction from the determined position.

5. (canceled)

6. (canceled)

7. The ultrasound imaging apparatus as claimed in claim 1, wherein the position determining unit comprises an input device for determining the position and the direction of the virtual viewing direction on the basis of a user input.

8. The ultrasound imaging apparatus as claimed in claim 7, wherein the input device is adapted to determine the position in the ultrasound data received from the ultrasound acquisition unit.

9. The ultrasound imaging apparatus as claimed in claim 1, wherein the imaging apparatus comprises a display unit for displaying the transformed ultrasound data in the virtual viewing direction.

10. The ultrasound imaging apparatus as claimed in claim 1, wherein the ultrasound acquisition unit is an external ultrasound acquisition unit located outside the patient's body or a catheter-based ultrasound acquisition unit.

11. The ultrasound imaging apparatus as claimed in claim 1, wherein the ultrasound data comprises a plurality of voxels each including an ultrasound measurement value and wherein the transformation unit is adapted to transform the ultrasound measurement values of the voxels in the field of view to voxels of the virtual field of view.

12. An ultrasound imaging method for providing ultrasound images of a patient (12), comprising the steps of:
receiving ultrasound data of a patient's body in a field of view,
determining a position within the patient's body,
transforming the ultrasound data in the field of view on the basis of the determined position to transformed ultrasound data in a virtual field of view having a virtual viewing direction different from the viewing direction of the field of view,
segmenting the ultrasound data in order to provide segmentation data,
determining the position on the basis of the ultrasound data, or on the basis of the segmentation data, or on the basis of X-ray data provided by an X-ray unit, and
determining the virtual viewing direction merely on the basis of anatomical features identified on the basis of the segmentation data or additionally the basis of the ultrasound data or the X-ray data.

13. A computer program comprising program code means for causing a computer to carry out the steps of the method as claimed in claim 12 when said computer program is carried out on a computer.
